(19) United States
(12) Patent Application Publication
(10) Pub. No.: US 2019/0083363 A1
(43) Pub. Date: Mar. 21, 2019

(54) COMPOSITIONS FOR THE REMINERALIZATION OF DENTIN

(71) Applicants: The Regents of the University of California, Oakland, CA (US); The University of Florida Research Foundation, Gainesville, FL (US)

(72) Inventors: Grayson Marshall, San Francisco, CA (US); Sally Marshall, San Francisco, CA (US); Hamid Nurrohman, San Francisco, CA (US); Kuniko Saeki, San Francisco, CA (US); Stefan Habelite, San Francisco, CA (US); Laurie Gower, Gainesville, FL (US)

(21) Appl. No.: 16/085,569

(22) PCT Filed: Mar. 16, 2017
(86) PCT No.: PCT/US2017/022799

§ 371 Date: Sep. 15, 2018

Related U.S. Application Data
(60) Provisional application No. 62/309,785, filed on Mar. 17, 2016.

(51) Int. Cl. A61K 6/027; A61K 6/08

(52) U.S. Cl. CPC A61K 6/0276; A61K 6/0008; A61K 6/08

(57) ABSTRACT

Provided herein are novel compositions for the restoration of demineralized tissues, such as dentin which has been demineralized by tooth decay. The remineralization agent comprises a bioactive ceramic component and a polyanionic macromolecular component, which may be admixed and applied to the target tissue and which will subsequently set to produce a remineralizing solid. The remineralizing solid will produce nanodroplets of mineral precursor solution, which such nanodroplets can infiltrate demineralized collagen matrices and which will form hydroxyapatite crystals therein by polymer-induced liquid precursor processes. The scope of the invention encompasses novel compositions and methods of treating demineralized tissues.

### CROSS-RELATED APPLICATIONS

This application claims the benefit of priority to U.S. Provisional Application Ser. No. 62/309,785, entitled “Biomimetic and bioactive dental restorative for in-situ remineralization,” filed Mar. 17, 2016, the contents of which are hereby incorporated by reference.

### STATEMENT REGARDING FEDERALLY SPONSORED RESEARCH OR DEVELOPMENT

This invention was made with government support under grant number R01 DE016849, awarded by the National Institutes of Health. The government has certain rights in the invention.

### BACKGROUND OF THE INVENTION

Teeth comprise dentin, a calcified tissue, overlaid with an outer layer of enamel. Teeth are under constant attack from chemical and physical forces, including bacterial-derived acids and mechanical wear, resulting in demineralization and weakening of enamel and the underlying dentin. Remineralization of dental tissues is the process of restoring minerals to the tooth structure. An effective remineralization treatment will restore the structure of the treated tissue and will reestablish mechanical properties like those of healthy tissues. While remineralization of enamel can be promoted by fluoride and other treatments, clinically effective methods of remineralizing dentin have not yet been achieved.

Remineralization of dentin has proven to be a difficult task due to the complex nature of this tissue, which is a biological composite mainly comprising collagen microfibrils reinforced with small crystals of apatite. In dentin, demineralization occurs in two regions, within collagen fibril helices and between collagen fibrils. Fortunately, in demineralized dentin the collagen matrix remains largely intact, potentially enabling the restoration of the tissue, if an effective remineralization agent could be developed.

Previous attempts to remineralize dentin by applying apatite precursors calcium and phosphate have been largely ineffective, because the minerals tend to precipitate on the outer surface of the collagen matrix rather than within and between the collagen fibrils, as in healthy dentin. Pioneering work by Gower and colleagues has elucidated a process by which native tissues such as bone or dentin are mineralized in vivo, called the polymer-induced liquid precursor (PILP) process. In PILP, an ion-sequestering species, such as a highly negatively charged protein concentrates mineral constituents, which induces a liquid-liquid phase separation, leading to the formation of nanodroplets containing hydrated amorphous mineral precursors. These nanodroplets then infiltrate the collagen fibrils through a mechanism hypothesized to occur by capillary action, and ultimately form mineral crystal structures that interpenetrate the supporting collagen matrix.

Experimental work with in-vitro model systems has shown that native PILP biomineralization processes can be mimicked with the PILP process to remineralize bone or dentin collagen matrices. However, the application of this process in a clinical context has not been achieved. Accordingly, there remains a need in the art for new technologies that can successfully apply the PILP processes in-vivo to promote remineralization of the collagenous matrices of living tissues for and the restoration of damaged teeth, bones, and other structures.

### SUMMARY OF THE INVENTION

Provided herein are novel compositions, methods, and systems for the remineralization of living tissues. In one aspect, the invention encompasses novel compositions which can be applied to demineralized tissues to promote their remineralization. In one aspect, the compositions of the invention comprise novel cement formulations which can be applied to damaged tissues such as dentin or bone tissue. In another aspect, the scope of the invention encompasses methods of remineralizing tissues such as demineralized dentin and bone by the application of the aforementioned compositions. In one aspect, the methods of the invention enable the treatment of dental pathologies, the restoration of damaged bone, and the production of implants. Further provided are novel kits which enable ready preparation of remineralization agents by practitioners.

### BRIEF DESCRIPTION OF THE FIGURES

FIGS. 1A, 1B, and 1C. FIGS. 1A, 1B, and 1C depict mineralization of collagen matrix by PILP. A demineralized tissue is depicted in FIG. 1A, comprising a bundle of collagen fibrils (101) with no mineral content. FIG. 1B depicts the infiltration of the collagen matrix by nanodroplets (102) comprising apatite precursors phosphate and calcium, wherein the nanodroplets aggregate (103) within the matrix. FIG. 1C depicts the collagen matrix after the nanodroplets have condensed and initiated the formation of apatite crystals (104) within intra- and inter-fibril spaces.

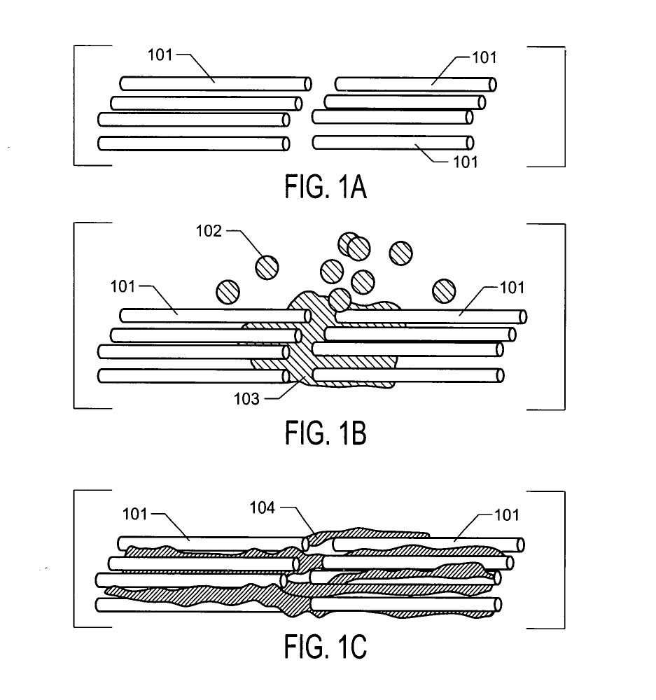

FIGS. 2A and 2B. FIG. 2A depicts the measured modulus of elasticity in demineralized dentin and in demineralized dentin treated for 14 days with a remineralizing composition of the invention, demonstrating remineralization of the treated dentin. FIG. 2B depicts the observed shrinkage in demineralized dentin slices treated with various compositions and in untreated dentin. A highly reduced degree of shrinkage was observed in dentin treated with remineralization composition BG40, demonstrating the remineralization capabilities of this composition.

### DETAILED DESCRIPTION OF THE INVENTION

Remineralization Agents.

The scope of the invention encompasses novel compositions for the remineralization of demineralized tissues, for example, demineralized dentin. The invention encompasses various remineralizing compositions made by mixing a bioactive ceramic material and a polyanionic macromolecular material. The remineralization agents of the invention can effectively cause or promote the remineralization of demineralized tissues in contact therewith. A demineralized tissue, as used herein, is any tissue having a mineral content that is below that typically observed in normal, healthy tissues. Exemplary tissues include dentin, bone, cementum, and enamel or any other tissues comprising a collagen fibril matrix. Exemplary demineralized tissues include dentin that has been exposed by tooth decay, and bone that has been demineralized by trauma, disease, aging, or malnutrition.

Bioactive Ceramic.

In the compositions of the invention, a first component is the bioactive ceramic material. The bioactive ceramic material comprises one or more species of bioactive glass, as known in the art ad being a biocompatible calcium-phosphosilicate composition which releases ions under physiological conditions, composed of varying amounts of silicates, calcium oxides, and phosphates.

In one embodiment, the bioactive glass comprises a composition comprising SiO₂, CaO, MgO, Na₂O, K₂O, and P₂O₅. In one embodiment, the bioactive glass comprises a SiO₂—CaO—MgO—Na₂O—K₂O—P2 O₅ material wherein the SiO₂ content is less than 57%. The solubility of the SiO₂—CaO—MgO—Na₂O—K₂O—P2 O₅ material may be increased by increasing the phosphate content and lowering the silica content. Mixtures of bioactive glasses with low solubility glasses can be used, in order to reduce the solubility (and bioactivity) of the material, as desired.

In one embodiment, the bioactive ceramic element comprises a bioglass. Bioglasses are commercially available formulations of bioactive glass comprising various amounts of silica, calcium oxide, phosphates, and sodium oxides. In one embodiment, the bioglass is 45S5. Other exemplary bioglass compositions which may be used in the practice of the invention include bioglass 8625, bioglass 42S5.6, bioglass 46S5.2, bioglass 49S4.9, bioglass 44S4.3, bioglass 453SF and Ceravital bioglasses.

In an alternative embodiment, the bioactive ceramic component may comprise a phosphate glass composition, having with little or no silica content. These glasses are highly soluble. In one embodiment, the bioactive ceramic component comprises a mixture of phosphate glass and silica-containing bioactive glass.

The characteristics of the bioactive ceramic material may be tuned in order to control the properties of solids formed therefrom. For example, decreasing the solubility of the bioactive ceramic element will generally reduce the setting time and increase the hydrolytic resistance of solids made therewith. Solubility may be tuned by varying the proportion of silicate and phosphate in the composition, with higher silica content imparting less solubility. Alternatively, dopants such as MgO and Na₂O may be added to reduce the solubility of the bioactive ceramic component. Various properties of the ceramic materials can be tuned, for example as described in U.S. Pat. No. 8,012,590, entitled “Glass/Ceramic Materials for Implants,” to Tomsia et al.

For the formation of the remineralization agent, the bioactive ceramic element will generally be used in a dry, powdered form. The particulate size may, for example, be in the range from <1 μm-20 μm. Particulate size will generally have a strong influence on the strength, hardness, modulus, and setting characteristics of the resulting solid, as known in the art. For the formation of solids, a higher proportion of smaller particles in the starting material generally corresponds to higher solid strengths, and an increased proportion of larger particles corresponded with a decrease in the viscosity of the unset cement, for example as described by Prentice et al, “The effect of particle size distribution on an experimental glass-ionomer cement,” Dent Mater (2005) 21, 505-510. Additionally, nano-sized materials may be included to modify the properties of the solid, for example as described for conventional GI cements by Moshaverinia et al., “A review of powder modifications in conventional glass-ionomer dental cements,” J Mater Chem 2011, 21, 1319-1328.

Polyanionic Macromolecular Material.

In the remineralization agents of the invention, the second component is a polyanionic macromolecular material. The polyanionic macromolecular material comprises one more species of a highly negatively charged, acidic polyanionic polymer or polypeptide. This material will form a cross-linked solid when combined with the bioactive ceramic element. In general, the polyanionic macromolecular material will have a sufficiently high charge density and molecular weight to sequester calcium and phosphate ions while stabilizing supersaturated ionic solutions and inhibiting classical nucleation of mineral crystals, thereby enabling the formation of nanodroplets of an amorphous mineral precursor phase. Such materials will produce nanodroplets, for example, when exposed to calcium phosphate containing solutions, at pH near 7.0.

The polyanionic macromolecular material may be comprised of a polymer of an acidic moiety that deprotonates under physiological conditions (near neutral pH), for example, polyanionic moieties of carboxylates, phosphates, phosphonates, or sulfates. Alternatively, the acidic moieties may be provided in deprotonated form (e.g., the sodium salts of the acid). The acids may comprise phosphorylated side groups.

In one embodiment, the polyanionic macromolecular material is a polyaspartic acid. In alternative embodiments, the polyanionic macromolecular material may comprises a polymer of any other polyanionic amino acid. In various embodiments, the polyanionic macromolecular element may comprise a polymer of a modified acid, such as a modified polyaspartic acid. For example, poly-L-aspartic has one carboxyl group per repeating unit. To increase the number of active groups per repeating unit, poly-L-aspartic acid can be grafted with species that increase the number of acidic moieties per unit. These functional groups can also provide for more protons to attack the bioactive ceramic element, and once deprotonated, provide for more ionic cross-linking sites to form bonds between cations and the polyanionic macromolecular backbone, causing faster setting and hardening of the resulting remineralization solid. For example, the aspartate or other acid residues of the polymer could be modified with acidic methacrylate monomers with phosphate functional groups. The carboxyl (—COOH) and phosphate (O—PO—[OH]₂) groups can etch enamel/dentin surfaces, promote adhesion, and stabilize amorphous mineral derived from both body fluids and mineral ion-released from the bioactive ceramic component, leading to improved kinetics of dentin remineralization.

Commercially available polyacids, such as polyaspartic acid, are often provided as sodium salts, however, in one implementation, sodium salts are not used because sodium reduces the reactivity of the polyanionic macromolecular material with the bioactive glass.

In another embodiment, the anionic acid residues of the polyanionic macromolecular material are modified with or otherwise comprise methacrylate groups (CH₂═C(CH₃)COO—) or other photo-curable moieties, allowing the remineralization agent to be set on demand by application of light or by auto-curing if appropriate chemical initiators, such as benzyl peroxide, are present in the mixture.

Polyanionic macromolecular materials of any molecular weight may be used, including monodisperse compositions and polydisperse mixtures of different molecular weights. For example, the polyanionic macromolecular material may comprise an average molecular weight of greater than 10 kDa, greater than 15 kDa, greater than 20 kDa, or greater than 30 kDa. In one embodiment, the molecular weight of the polyanionic macromolecular material is between 20 to 30 kDa, for example, having a molecular weight of 23-27 kDa.

In some embodiments, the polyanionic macromolecular element of the invention comprises a negatively charged polypeptide. In one embodiment, the polyanionic macromolecular material comprises osteopontin protein. Osteopontin is an extensively phosphorylated acidic glycoprotein found in bone, milk and other biomaterials. Osteopontin is a SIBLING (small integrin-binding ligand, N-linked glycoprotein) protein, which are generally highly charged and intrinsically disordered proteins. In the practice of the invention, the osteopontin may be extracted from biological materials or may be recombinantly produced, and may be derived from any species, including humans, bovines, and other animal species. In one embodiment, the osteopontin comprises OPN-10, a polydisperse mixture of purified bovine osteopontin proteins derived from milk, available commercially as LACPRODAN™ (Arla Foods, Denmark).

In alternative embodiments, other SIBLING proteins may be used, including bone sialoprotein, dentin matrix protein 1, dentin sialophosphoprotein, and matrix extracellular phosphoglycoprotein. Other charged glycoproteins, GAGS, or polysaccharides may be used as well.

In the formation of the remineralization agent, the polyanionic macromolecular material may be utilized in a liquid form, comprising a concentrated solution of the polyanionic macromolecular in water, calcium phosphate solution or other appropriate solvent. Exemplary concentrations are in the range of 10-200 mg/l. Alternatively, the polyanionic macromolecular material may be utilized in a dry form.

Ancillary Components.

The bioactive ceramic element and/or the polyanionic macromolecular material may be augmented with additional species which affect the formation of the remineralization solid, or which enhance the functionality of the remineralization agent. For example in one embodiment, tartaric or maleic acid are added to the polyanionic macromolecular material to improve its reactivity and shelf file. In one implementation, cross-linking species may be added to the bioactive ceramic component and/or the polyanionic macromolecular material, in order to speed the kinetics of solid formation or in order to increase the mechanical strength of the resulting solid. For example, cross-linking resin species known in the art, such as photo-curable resins or chemically-induced cross-linkers may be used. In another embodiment, fluoride is included in the bioactive ceramic component and/or the polyanionic macromolecular element, such that the resulting remineralization agent releases fluoride, which promotes the remineralization of enamel. For example, fluorapatite crystals may be present in the silica matrix of the bioactive ceramic component. In one embodiment, polyacrylic acid is added to the polyanionic macromolecular component to reduce the set time of the remineralization agent and increase its mechanical strength. In one embodiment, pepotoids, comprising cation-sequestering peptides and as described in United States Patent Application Publication Number 2015/0174197, entitled “Peptides useful for the mineralization of apatite,” by Zuckerman et al., may be used in the remineralization agent.

Remineralization Agent.

The remineralization agent of the invention comprises a mixture of a bioactive ceramic material and an polyanionic macromolecular material. The remineralization agent may exist in two forms. Upon mixing the bioactive ceramic material, the polyanionic macromolecular material, and solvent, the remineralization agent may temporarily exist in an unsolidified form. In this states, and before setting, the composition will be referred to herein as a “remineralization solid precursor.” In this form, the composition comprises a slurry or viscous paste. In this phase, protons from the polyanionic macromolecular are attacking the phosphosilicate bioactive ceramic material, liberating ions that facilitate cross-linking of the polymer backbones.

After a period of time, for example, minutes following the admixture of the bioactive ceramic material and the polyanionic macromolecular material, a solid is formed by ongoing gelation processes. In the set, or solidified form, the mineralization agent will be referred to herein as a “remineralization solid.” The remineralization solid comprises a gel made up of bioactive ceramic components intercalated within an polyanionic macromolecular matrix comprising polyanionic macromolecular s cross-linked via ionic bridges and adhered to surrounding tissues by the anionic moieties of the polymers. Undissolved ceramic material is also entrapped within this matrix. Depending on the starting materials and proportions thereof in the mixture, the remineralization solid may comprise a soft or hard gel. Generally, harder solids are preferred, for example, solids having an elastic modulus of 2-10 GPa. “Setting,” as used herein may be defined as substantial solidification of the material, for example as determined by any standard measure thereof, such as the Gilmore needle test.

Without being bound to any particular theory as to the mode of action of the claimed materials, it is believed that, in vivo, formed adjacent to or in contact with demineralized tissues, nanodroplets derived from the remineralization solid (cement) will move into the collagen network of the demineralized tissue and will adsorb to collagen fiber surfaces at specific binding domains. Nanodroplets will release calcium and phosphate ions into the gap zones, which are spaces between collagen molecules within the fibrils and the interstitial spaces between collagen triple helices. The high content of mineralizing ions within the fibrils leads to the formation amorphous calcium phosphate (ACP). Over a period of 24 to 48 h, ACP will transform into crystal structures, for example hydroxyapatite (HAP) crystal platelets formed by the precipitation of calcium and phosphate. HAP crystals measure about 10 to 20 nm in width and length and are oriented with their basal plane in parallel to the fibril long axis. By this manner, the remineralization solid will restore the hydroxyapatite platelets and other crystal structures found in healthy mineralized tissues and recover most of the elastic properties of the tissue.

The remineralization agent may be formed using varying ratios of bioactive ceramic material and polyanionic macromolecular material. The ratio of bioactive ceramic component to polyanionic macromolecular may range from 10:90 to 90:10, for example in the range of 40:60 to 60:40, with ratios expressed by weight.

In one embodiment, the mineralization agent is formed by the combination of bioglass 45S5 and polyaspartic acid, mixed at a ratio of between 90:10 and 40:60 by weight, for example at a ratio of about 60:40. In another embodiment, the mineralization agent is formed by the combination of bioglass 45S5 and osteopontin, for example, OPN-10, mixed at a ratio of between 90:10 and 40:60 by weight, for example at a ratio of about 60:40. For these exemplary compositions, setting times of less than ten minutes were observed.

If the polyanionic macromolecular material is provided as a solution, the solvent contained therein will generally provide sufficient liquid to facilitate the necessary chemical reactions and provide easy mixing and processing of the mixture. If the polyanionic macromolecular material is provided as a powder or otherwise in dry form, small amounts of water or other solvents will be introduced to the admixture at the time the remineralization solid precursor is formed. Generally, the mixture should contain only small volumes of water, sufficient to form a thick paste or slurry, for example water at 10-30% by weight.

In one embodiment, the remineralization agent of the invention comprises what will be referred to herein as an “augmented cement,” which means a conventional cement formulation which has been augmented with the bioactive ceramic material and/or the polyanionic macromolecular components of the invention. By adding one or both of these components, the functionality of the conventional cement formulation is augmented with remineralization abilities. For example, in the dental context, the conventional cements may comprise Ca and Al oxides, such as fluorosilicate or aluminosillicate glass powder, combined with polyionic liquid containing polyacrylic acid with various modifying co-monomers such as tartaric, itaconic acid. Exemplary conventional cements include FUJI-IX™ (GC-America, Illinois, US), BIOCEM™ (NuSmile, TX, USA), and BIODENTINE™ (Septodont, France). In one embodiment, the conventional cement is augmented with a polyanionic macromolecular component, such as polyaspartic acid or OPN-10. The polyanionic macromolecular component may be added to the conventional cement constituents at proportions, for example, of between 5 and 50%. For example, in one embodiment, the augmented cement of the invention comprises a standard cement admixed with polyaspartic acid at 20%-40%, by weight.

Kits of the Invention.

In one aspect, the scope of the invention encompasses kits, wherein the kits comprise a combination of separately packaged bioactive ceramic material and polyanionic macromolecular solution (or powdered forms thereof, provided with solvents or directions for combining with water or other common solvents). Such kits may be provided to practitioners and will enable the facile production of remineralization by mixing measured aliquots of the two components. In one embodiment, the kit of the invention comprises one or more aliquots of bioactive ceramic material and one or more aliquots of a polyanionic macromolecular solution, wherein an aliquot of each can be mixed to form a remineralization solid precursor. The kits may further comprise elements that facilitate the ready production of remineralization solid precursors, including: packaging, instructions, mixing implements, measurement devices (e.g. scoops); dispensers (e.g. syringes or dropper bottles), and vessels, as known in the art.

Methods of the Invention.

The scope of the invention extends to methods of using the various compositions described herein. In a first embodiment, the method of the invention comprises a method of producing a remineralization agent by mixing appropriate amounts of bioactive ceramic component and polyanionic macromolecular component. The mixing may be accomplished by the means of hand tools, for example by amalgam mixing spatula, mixing tips, or by automated devices such as those utilized in the mixing of conventional GI cements. If the polyanionic macromolecular material is in a dry or powdered form, the method further encompasses the inclusion of solvents such as water in the admixture.

In one embodiment, the invention encompasses a method of making a medicament for the treatment of demineralized tissues, comprising the formation of a remineralization solid precursor.

In an another embodiment, the method of the invention comprises a method of treating or remineralizing a demineralized tissue by the application of a remineralization agent on the surface of a demineralized tissue. It will be understood that, for convenience, reference is made herein to the application of a remineralization agent to the surface of a treated tissue, but that such reference will also encompass any placement of the demineralization agent in sufficient proximity and fluid connection with the demineralized tissue that nanodroplets derived from the remineralization solid can access the treated tissue. In a first embodiment, the method of the invention comprises a method of treating or remineralizing a demineralized tissue by the application of a remineralization solid precursor, wherein such mixture is applied directly onto the surface of a demineralized tissue. In a second embodiment, the method of the invention comprises the treatment or remineralization of a demineralized tissue by the formation of a remineralization solid on the surface of the demineralized tissue. The treated tissue may be that of a human patient, test animal, or veterinary subject. The tissue may comprise any demineralized tissue, including dentin or bone. Treatments may comprise therapeutic treatments to restore damaged tissues or preventative treatments to maintain a highly mineralized state in treated tissues.

In one aspect, the methods of the invention are applied in a dental context, for the treatment of caries, lesions, and demineralized dentin exposed by tooth decay or by mechanical means (e.g. by drilling or by dental burs). In the methods of the invention, the bioactive ceramic component and polyanionic macromolecular component are mixed and may then be applied to the target dentin, for example by hand tools such as a cement spatula or brush.

In one embodiment, the invention encompasses a method of using a remineralization agent in the treatment of demineralized tissue.

In some embodiments, the hydrolytic stability of the remineralization solid formed on the dentin is such that it will not persist for long periods in the environment of the mouth without degrading. To preserve such compositions, a protective cap may be formed over the applied remineralization agent, for example, after it has partially or fully set, in order to protect it from the hydrolytic oral environment. For example, a cap comprising a resistant material, such as dental amalgam, flowable resin, resin composite, standard glass ionomeric cement, or polymeric species may be used. The cap may comprise a pre-manufactured object that is adhered onto or over the remineralization agent, or it may be a structure that is formed in-situ by depositing unset material onto and around the applied remineralization agent, sealing it off from the oral environment. In one embodiment, the cap is a permanent cap, wherein the remineralization agent is deposited as a liner onto the treated area and then is covered by a cap of more resistant material intended to remain in place for long periods of time. In an alternative embodiment, the cap is a temporary cap that may be removed after a period of time (e.g. within days, weeks, or months of application) so that the previously-applied remineralization solid may be removed and replaced with fresh material, for example in a treatment regime comprising a series of repeated applications.

In one embodiment, the treated tissue is bone. The compositions of the invention may be applied as bone grafts, bone substitute, or bone patches, for example in the treatment of fractures, or other injuries to bone or to bone tissues degraded by trauma, disease, malnutrition, or aging. In such an implementation, the remineralization solid precursor may be applied to bone surface that has been exposed by surgical means or may be injected to the bone surface by a needle. In one embodiment, the delivery of the remineralization solid precursor to the target tissues may be enhanced by the application of ultrasound or other energetic treatment.

Synthetic Scaffolds.

The compositions and methods of the invention have been described with respect to the remineralization of demineralized tissue. In a related implementation, the compositions described herein may be combined with a synthetic matrix material. The compositions of the invention can facilitate the mineralization of such synthetic scaffolds. Scaffolds include any biocompatible, mineralizable material, including porous, mesh, or fibrous materials. Exemplary scaffold materials include biodegradable polymers, such as polyesters such as poly(lactic acid), poly(glycolic acid) and their copolymers, as well as ceramics such as hydroxyapatite and tricalcium phosphate. Materials such as porous zirconia hydroxyapatite and polycaprolactone may also be used. Synthetic scaffolds may further encompass biomaterials formed through bioengineering methods. Scaffolds which are osteogenic and/or biodegradable may be used.

In a related method, the combined mineralizing compositions and scaffolds can be implanted at the site of tissue (e.g. dentin or bone) defects to fill spaces caused by trauma or disease, for example to fill lesions caused by tumor, periodontal, periapical infection, trauma or extraction.

Implantable Objects.

In another aspect, the scope of the invention extends to remineralization solids that are formed ex-vivo and which are then implanted at the treatment site. For example, remineralization solids in the form of as particulates, beads, films, or patches may be formed ex-vivo and may then subsequently be deposited onto the surface of demineralized tissues. For example, particulates such as beads may be intermixed with unset conventional cements, or may be applied to form a liner under such conventional cements.

### EXAMPLES

### Example 1. Set Time Evaluation

The setting time of various remineralization agent compositions was tested. Bioactive glass (Bioglass 45S5) was mixed with polyaspartic acid (MW˜23-27 kDa) at ratios of 9%, 16%, 19.2%, and 37.5% weight bioactive glass: weight polyaspartic acid. The formulations were made using 40 mg bioglass, 22 microliters of water, and varying amounts of polyaspartic acid (4, 8, 12, and 24 mg polyaspartate powder). Set time was assessed by the Gilmore needle-test. Higher proportions of bioactive glass resulted in less soluble compositions having shorter set times. At 9% bioactive glass, the material did not set. At 16%, the material took longer than 10 hours to set. At 19.2% bioactive glass, set time was 16 minutes, and at 37.5% bioactive glass, set time was 5-8 minutes. Similar initial results were found when OPN-10 was used in place of polyaspartic acid.

### Example 2. Remineralization Agents

The ability of various compositions to remineralize depleted dentin was evaluated. The compositions included a 60:40 mixture of bioactive glass (Bioglass 45S5) and polyaspartic acid (MW˜23-27 kDa), termed “BG40.” Also tested were unmodified BIOCEM™ cement, a mixture of 80:20 BIOCEM™ cement and polyaspartic acid (“BIOCEM20”), a mixture of 60:40 BIOCEM™ cement and polyaspartic acid (“BIOCEM40”), unmodified FUJI-1™ and unmodified FUJI-IX™ glass ionomer cements.

Human molars were sectioned laterally, providing dentin slices that were demineralized by treatment in CaHPO₄ and acetic acid solution for 66 hours to remove native hydroxyapatite. A coating of test composition was applied onto the top portion of each dentin slice. A control treatment comprised dentin without a cement top coating (“DEMIN”). These samples were capped with flowable resin. Samples were embedded in epoxy. The epoxy-embedded dentin slice was placed with the lower, exposed surface of the dentin immersed in a simulated body fluid solution, such that the pulpal chamber was in contact with the simulated body fluid.

After 14 days of treatment, substantial mineralization of the BG40-treated dentin was observed visually.

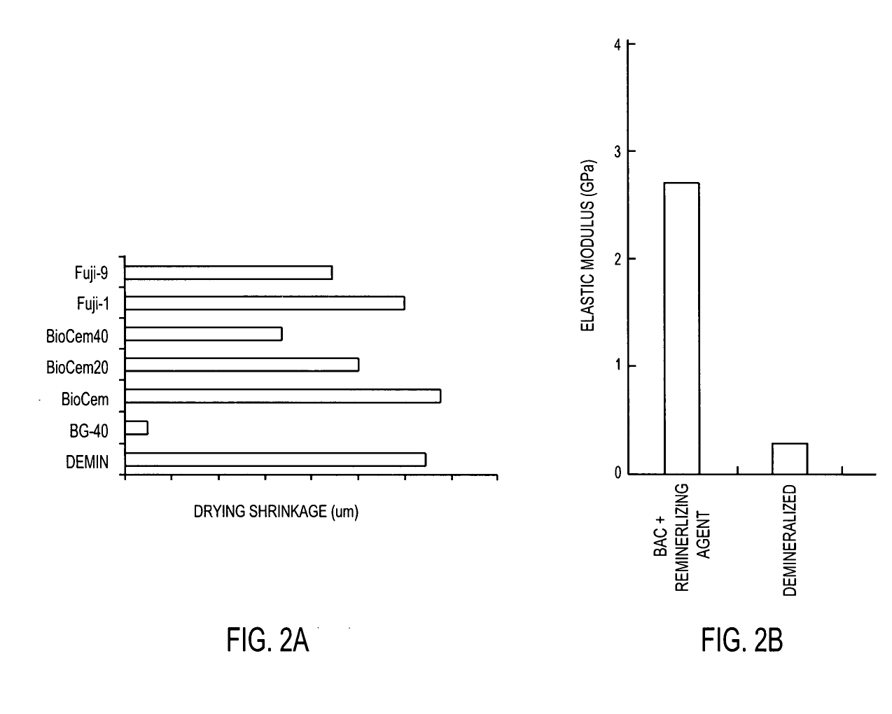

Nano-indentation measurements were made to determine the elastic modulus of the treated dentin slices. Elastic modulus in the BG40 treated-dentin was high (FIG. 2B, BG40 labeled “Bac+Mineralizing Agent” and untreated control labeled “Demineralized”), while a low elastic modulus was observed in the control treatments and dentin treated with other formulations, demonstrating mineralization of the depleted dentin by the BG40 treatment but not by unmodified conventional cements.

Vertical shrinkage of the dentin slices, which correlates with the degree of mineralization, was assessed by light microscopy (as described in Burwell et al., “Functional Remineralization of Dentin Lesions Using Polymer-Induced Liquid-Precursor Process,” PLOS 2012 7(6): e38852. doe: 10.1371/journal.pone.0038852) (FIG. 2A). Substantial shrinkage was observed in the control treatment and in dentin treated with unmodified conventional cements as well as the mixture of 80:20 BIOCEM™ cement and polyaspartic acid. Substantially less shrinkage was observed in the dentin treated with BG40. Intermediate shrinkage was observed in the dentin treated with a mixture of 60:40 BIOCEM™ cement.

These results demonstrate that while conventional cements were lacking in their ability remineralize dentin, the remineralizing capabilities of BG40 were excellent.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1-31. (canceled)

32. A remineralization agent, comprising
an admixture of
a bioactive ceramic material; and
a polyanionic macromolecular material.

33. The composition of claim 32, wherein
the bioactive ceramic material comprises a bioglass.

34. The composition of claim 33, wherein
the bioglass comprises 45S5.

35. The composition of claim 34, wherein
the bioactive ceramic material comprises SiO₂, CaO, MgO, Na₂O, K₂O, P₂O₅, and wherein the SiO₂ content is less than 57% by weight.

36. The composition of claim 32, wherein
the bioactive ceramic material comprises a phosphate glass.

37. The composition of claim 32, wherein
the particulate size of the bioactive ceramic component is between 1 and 20 μm.

38. The composition of claim 32, wherein
the polyanionic macromolecular material comprises a polymer of polyanionic acid.

39. The composition of claim 38, wherein
the polyanionic acid is a polyanionic moiety of carboxylate, phosphate, phosphonate, or sulfate.

40. The composition of claim 38, wherein
the polymer of polyanionic acid comprises polyaspartic acid.

41. The composition of claim 38, wherein
the polyanionic macromolecular material has an average molecular weight of greater than 10 KDa.

42. The composition of claim 38, wherein
the polyanionic macromolecular material has an average molecular weight of 20-30 KDa.

43. The composition of claim 32, wherein
the polyanionic macromolecular component comprises a polypeptide.

44. The composition of claim 43, wherein
the polypeptide comprises a SIBLING protein.

45. The composition of claim 44, wherein
the SIBLING protein is selected from the group consisting of osteopontin, bone sialoprotein, dentin matrix protein 1, dentin sialophosphoprotein, and matrix extracellular phosphoglycoprotein.

46. the composition of claim 44, wherein
the sibling protein comprises purified bovine osteopontin.

47. The composition of claim 32, wherein
the proportion of the bioactive ceramic material to polyanionic macromolecular material is between 10 and 90% by weight.

48. The composition of claim 47, wherein
the proportion of the bioactive ceramic material to polyanionic macromolecular material is between 40 and 60% by weight.

49. The composition of claim 32, further comprising
a cross-linkable resin component.

50. The composition of claim 32, further comprising
fluoride.

51. The composition of claim 32, further comprising
maleic acid or tartaric acid.
